(19) United States
(12) Patent Application Publication
(10) Pub. No.: US 2012/0045483 A1
(43) Pub. Date: Feb. 23, 2012

(54) Mannitol and/or Proline for Prevention and Treatment of Ageing Related Symptoms

(75) Inventors: Gijsbertus van der Horst, Rhoon (NL); Jan Hoeijmakers, Zevenhuizen (NL); Ingrid van der Pluijm, Papendrecht (NL)

(73) Assignee: ERASMUS MC, Rotterdam (NL)

(21) Appl. No.: 13/226,242

(22) Filed: Sep. 6, 2011

Related U.S. Application Data
(63) Continuation of application No. 12/300,738, filed on Feb. 5, 2009, now abandoned.

(30) Foreign Application Priority Data
May 15, 2006 (EP) 06113951.5

(51) Int. Cl. A61K 31/401; A61K 9/00; A61P 7/06; A61P 3/00; A61P 17/00; A61P 25/00; A61K 31/047; C07C 31/26

(52) U.S. Cl. 424/400; 514/738; 514/423; 568/852

(57) ABSTRACT

The current invention provides new methods and means for the prevention and treatment of ageing-related symptoms and diseases. The invention discloses Mannitol 5 and/or proline containing compositions that are particularly useful for the treatment of premature ageing related symptoms in mammalian subjects suffering from genetic defects in DNA damage response and genome maintenance pathways. Humans suffering from Cockayne syndrome (CS), Xerodema pigmentosum (XP), combined XPCS, trichothiodystrophy (TTD), COFS (cerebro-oculo-facio-skeletal syndrome), 10 XFE disorder (Xpf-Erccl syndrome), Bloom Syndrome (BS), Werner Syndrome (WS), Ataxia telangiectasia (AT), Fanconi Anemia (FA), Hutchinson Guilford Progeria (HGP) may be treated with pharmaceutical compositions comprising mannitol and/or proline according to this invention.

### FIELD OF THE INVENTION

The invention relates to the field of medicine and in particular to the field of DNA repair syndromes and ageing.

### BACKGROUND OF THE INVENTION

Ageing is a syndrome of deleterious changes in the body that are progressive, universal and mostly irreversible. Ageing in humans is often accompanied with several diseases and syndromes that increase in frequency with age, such as arthritis, osteoporosis, heart disease, cancer, Alzheimer's Disease, etc. Ageing is thought to be at least in part a result of accumulating DNA damage in the genome, due to insufficient repair and faulty repair of coding and regulatory sequences in the genome.

DNA repair is constantly active in living cells and protects the genome from DNA damage and harmful mutations and ensures maintenance of the genome, required for correct replication and transcription of genes. In human cells, both normal metabolic activities (notably oxidative respiration, producing reactive oxygen species) and environmental factors (such as UV- and X-rays, numerous genotoxic chemicals) cause many types of lesions in the DNA including oxidative DNA damage. This together with spontaneous hydrolysis are believed to result in as many as 10,000 to 50,000 lesions per cell per day, These lesions cause structural damage to the DNA molecule, such as strand breaks, inter- and intrastrand crosslinks, depurination, depyridination and formation of bulky adducts, all of which can dramatically affect gene transcription and subsequent translation processes as well as DNA replication, Consequently, the DNA repair process must be constantly operating, to correct rapidly any damage in the DNA structure.

As cells age, DNA damage accumulates in the genome. An ageing cell then suffers one of three possible fates: an irreversible state of dormancy, known as cellular senescence, cell death, (mostly by apoptosis or programmed cell death) or induction of mutations that may trigger carcinogenesis. Most cells in human tissues are terminally differentiated and during aging the fraction of senescent cells increases, DNA damage can interfere with gene expression by preventing transcription of RNA from DNA, whereas mutations usually can result in transcription that produces proteins with diminished or altered functionality. Mutations that are not lethal to a cell are more likely to be perpetuated in dividing cells, DNA damage rather than DNA mutation is posited as a cause of aging.

Several potent excision repair systems operate on the basis of damage or mutilation occurring in only one of the two strands of the DNA double-helix such that the undamaged strand can be used as a template to repair the damaged strand. The damaged area of the injured strand is cut-away (excised) and a new strand (or a single nucleotide) is resynthesized. Even the simplest repair usually involves large protein complexes comprising proteins with different enzymatic activities.

There are three general categories of excision-repair pathways; (1) Base Excision Repair (BER, which repairs subtle base damage) (2) Nucleotide Excision Repair (NER, for repairing helix-distorting DNA strand damage excising a single strand region of 2-30 bases in length) and (3) MisMatch Repair (MMR, for repairing mispaired bases due to replication errors).

Base Excision Repair (BER) primarily repairs damage due to hydrolysis, alkylation (usually methylation) or oxidation of nucleotide bases. Subtle alterations of a base may not impede transcription or replication but such lesions are often miscoding leading to mutations.

Nucleotide Excision Repair (NER) repairs damage that generally distorts the helical conformation of DNA. This includes damage affecting more than one nucleic acid base, for instance cross-links between purines & the deoxyribose-phosphate backbone due to the hydroxyl radicals and pyrimidine dimers (two covalently-bonded adjacent pyrimidines) caused by ultraviolet light. Carcinogenic lesions like those caused by aflatoxins or cisplatinum (which form bulky DNA adducts and inter- and intrastand crosslinks respectively) are predominantly repaired by NER. Many steps and more than 25 proteins are involved in recognizing the type of damage unwinding the DNA around the lesion, damage verification, excision of the lesion in the form of a 24-32 oligonucleotide and in gapfilling DNA synthesis and ligation of the final nick.

There are two subtypes of NER: Global-Genome Nucleotide Excision Repair (GG-NER) and Transcription-Coupled Nucleotide Excision Repair (TC-NER, when also transcription-blocking, non-NER lesions are included this pathway is designated trancription-couplcd repair or TCR). The GG-NER pathway operates genome wide and is very important for preventing mutations. However, for some types of less-distorting lesions this process is rather slow. GG-NER recognizes and eliminates distorting strand defects using the concerted action of a set of enzymes including XP protein(complexe)s, which were identified by virtue of naturally occurring human inherited syndromes; notably the prototype DNA repair disorder Xeroderma Pigmentosum (XP) characterized by sun (UV) hypersensitivity, cutaneous pigmentation abnormalities and a >2000-fold elevated skin cancer predisposition in sun-exposed parts. The XPC protein complex is the main DNA damage detector and initiator of the GG-NER reaction, followed by local unwinding of the DNA around the lesion by the XPB and XPD helicase subunits of the TFIIH complex so that other NER proteins can verify the damage and incise the damaged strand on both sides of the lesion and DNA polymerases and other replication factors can resynthesize the lost DNA sequence. Transcription Factor IIH (TFIIH) functions in normal transcription as well as unwinding DNA for repair. The TC-NER subpathway focuses on repair of lesions in the transcribed strand of active genes that actually block transcription. This system thus enables recovery of the vital process of RNA synthesis and promotes cellular survival from DNA damage. TC-NER and the broader TCR pathway include CS proteins which were identified as being defective in individuals suffering from the severe neuro-developmental photosensitive disorder Cockayne Syndrome (CS). CS proteins are thought to aid in displacement of the stalled RNA polymerase to allow NER enzymes to access the damaged DNA. The TFIIH helicase complex does helic unwinding followed by other NER factors that take care for lesion verification, excision and gap-filling DNA synthesis as outlined above for GG-NER.

MisMatch Repair (MMR) corrects errors made during DNA copying, such as the mispairing of an adenosine base with a guanosine. MMR can correct A-C & T-C mismatches more efficiently than G-A & G-T mismatches. Proteins involved in MMR when mutated cause the human syndrome HNPPC; hereditary or familial non-polyposis colorectal cancer.

In humans a number of DNA repair and maintenance pathways have been uncovered, many of which by virtue of naturally occurring mutations in genes involved in these pathways that cause a specific syndrome, hereditary DNA repair disorders, Several of these syndromes comprise symptoms of premature ageing (references: Hoeijmakers et al. 2001, Nature 411:366-74; Hasty et al. 2003, Science 299:1355-9; Mitchell et al. 2003, Curr Opin Cell Biol 15:232-40; Martin 2005, Cell 120:523-32).

Defects in the Nucleotide Excision Repair (NER) mechanism are responsible for several genetic disorders, including:

Xeroderma pigmentosum (mutations in XPA, XPB, XPC, XPD, XPE, XPF and XPG genes): hypersensitivity to sunlight/UV, resulting in increased skin cancer incidence and sun-induced pigmentation alterations.

Cockayne syndrome (mutations is CSA and CSB genes): hypersensitivity to UV and chemical agents, severe, progressive neurodevelopmental dysfunction reminiscent of premature ageing. This disease can also occur in combination with XP

Trichothiodystrophy: comprising symptoms as a sensitive skin, brittle hair and nails, mental and physical retardation, which often accompanies the latter two disorders, suggests increased vulnerability of developmental neurons, and is consistent with features of premature ageing.

Other DNA repair disorders in other DNA repair or damage response pathways include:

Werner's syndrome (mutations WRN gene): displaying premature aging and retarded growth.

Bloom's syndrome: sunlight hypersensitivity, high incidence of malignancies (especially leukemias).

Ataxia telangiectasia (ATM gene); characterized by neuronal ataxia and telangiectasias, increased hematological malignancies and neuronal degeneration. At the cellular level hypersensitivity to ionizing radiation and some chemical agents.

Fanconi anemia (caused by mutations in at least 12 distinct FA-genes); developmental abnormalities with pancytopenia and increased frequency of acute myeloid leukemia. At the cellular level hypersensitivity to DNA crosslinking agents.

To the same category belongs also the prototype premature aging disorder Hutchinson-Gilford Progeria (HGP), in which patients display early cessation of growth, baldness at the age of 2, degenerative processes in the skin, muscle and bone and often fatal atherosclerosis. The disease is due to specific pointmutations in the gene for nuclear laminA, causing nuclear disorganization, genome instability and likely also disturbance of DNA damage response systems.

The above mentioned syndromes are often referred to as “segmental progerias” or “accelerated aging diseases” because affected individuals suffer from aging-related symptoms and diseases in at least some, mostly multiple (but not all) organs/tissues at an abnormally young age. Hence, many but not all aspects of ageing are accelerated in these conditions. Thus far there is no therapy available for patients suffering from DNA repair and premature ageing syndromes, other than avoiding the causes of DNA damage such as UV irradiation (exposure to sunlight). The current invention provides new insights in the possible treatment or amelioration of the premature ageing conditions in individuals suffering from genetically inherited DNA repair deficiencies and may also be applied to symptoms of natural ageing.

### SUMMARY OF THE INVENTION

The current invention provides new methods and means for the prevention and treatment of ageing-related symptoms and diseases. Ageing related symptoms may comprise also cancer and ischemia-reperfusion damage (e.g. as in organ transplantation, and cardiovascular disorders), processes in which oxidative damage is implicated. In particular the invention provides compositions comprising mannitol or proline, preferably both mannitol and proline, for use as a medicament. A synergistic effect may be achieved when mannitol and proline are administered in combination, either simultaneously or sequentially. According to the current invention mannitol and/or proline are particularly useful for the treatment of ageing related symptoms in mammalian subjects suffering from genetic defects in DNA damage response/repair (or genome maintenance) pathways, most preferably human subjects. Humans suffering from Cockayne syndrome (CS), Xeroderma pigmentosum (XP), combined XPCS, trichothiodystrophy (TTD), COFS (cerebro-oculo-facio-skeletal syndrome), XFE disorder (Xpf-Erccl syndrome), Bloom Syndrome (BS), Werner Syndrome (WS), Ataxia telangiectasia (AT), Fanconi Anemia (FA), Hutchinson Guilford Progeria (HGP) may be treated with mannitol and/or proline according to this invention. These syndromes result in impaired genome maintenance, and increased cell death or replicative senescence and give rise to a premature, accelerated and enhanced segmental ageing phenotype in addition to increased cancer incidence in a large fraction of these conditions. In addition the invention is relevant for other applications in which oxidative damage is involved most notable ischemia-reperfusion as applies to cardiovascular disease and organ transplantation. The current invention pertains to a method treatment with compounds or mixtures of compounds capable of preventing, delaying, inhibiting or curing premature ageing symptoms caused by genome maintenance defects, including cancer predisposition.

### DETAILED DESCRIPTION OF THE INVENTION

Mannitol or hexane-1,2,3,4,5,6-hexyl (C₆H₈(OH)₆) is an alcohol and a sugar, or a polyol; it is similar to xylitol and is a sorbitol isomer. At high doses, mannitol may be used as an osmotic diuretic agent and a weak renal vasodilator. Mannitol may further be used to reduce intracranial pressure in the cranium and to treat patients with oliguric renal failure. It may be administered intravenously or orally, and is filtered in the kidney. Given as a hypertonic solution, it increases distal tubule delivery of Na+ and water, resulting in increased urine formation. At high doses, mannitol can also be used to open the blood-brain barrier by temporarily shrinking the tightly coupled endothelial cells that make up the barrier. This makes mannitol useful for delivering various drugs directly to the brain (e.g. in the treatment of Alzheimer's disease). Mannitol is also used as a sweetener for people with diabetes. In doses larger than 20 g, mannitol acts as a laxative, and is sometimes sold as a mild laxative for children. Like other polyols, mannitol has a mild antioxidant effect by scavenging off free hydroxyl radicals, preventing oxidative damage from reactive oxygen species (ROS).

L-Proline is one of the twenty amino acids which are used in living organisms as the building blocks of proteins. The other nineteen units are all primary amino acids, but due to the (3-carbon) cyclic sidechain binding back to the nitrogen of the backbone, proline lacks a primary amine group (—NH₂). The nitrogen in proline is properly referred to as a secondary amine. Proline is a major amino acid found in cartilage and has been implicated in repair of muscle tissue, connective tissue and skin damage. The antioxidant properties of proline have been documented widely, in plants and microorganisms and mammals (reviewed in Matysik et al., Current Science, vol 82, no. 5, 2002).

The current invention provides compositions comprising a source of mannitol or proline, preferably both mannitol and proline. The compositions of the invention may be of any type. The compositions of the invention are preferably pharmaceutical compositions, nutraceutical compositions, food and beverage compositions, or cosmetic compositions. Mannitol and proline in particular have well documented anti-oxidant properties. However, the anti-oxidant effect of these compounds is likely not the sole mechanism of action of these compounds according to this invention. Other compounds with more pronounced anti-oxidant properties such as vitamin C and E did not yield the positive results of mannitol and/or proline when tested under similar conditions. Moreover, when testing effective concentrations of mannitol and/or proline, (ranged between 0.1 μg/ml and 10 μg/ml as measured in the serum), no or negligible AOC (anti-oxidant capacity) could be measured in vitro. Hence, other properties of mannitol and proline apart from anti-oxidant properties contribute significantly to their beneficial effect in individuals suffering from genetic defects in a DNA damage response/repair pathway and/or (premature) ageing syndromes. A mere administration of anti-oxidant compounds does not suffice for use according to this invention.

The present inventors are the first to demonstrate an in vivo protecting role for mannitol and/or proline at relatively low concentrations as exemplified herein. This was established using a mouse model for premature ageing phenotype (Csb/Xpa double knock out, see example 1). The skilled person would generalize this demonstrated effect of mannitol and/or proline in this mouse model for any (natural) ageing related symptoms, for other premature ageing phenotypes and/or for phenotypes associated with genetic defects in a DNA damage response/repair pathway.

In order to be effective in vivo, compositions according to the invention preferably are controlled release formulations, in particular slow release formulations that provide a constant source of mannitol and/or proline. Without wishing to be bound by theory, the rationale behind slow release formulations is the rapid clearing of both mannitol and proline from the body of a subject. The inventors have unexpectedly found that relatively low but constant sources of mannitol and/or proline are sufficient for the composition to be fully effective. In contrast, higher single dose administrations were proven less or even ineffective and were rapidly cleared from the tissues and bloodstream of treated subjects. This observation is also in agreement that the anti-oxidant effect per se is not sufficient for use according to this invention.

Secondly, subjects suffering from premature ageing syndromes, in particular CS patients, suffer from a lack of appetite, stomach acid reflux, frequent nausea and vomiting, and are generally malnourished and/or thin. Mannitol administered in higher concentrations, in particular when given as a single dose, may exacerbate the state of malnourishment by its well known properties as an osmotic diuretic. A controlled, slow release formulation will avoid problems of both frequent intake and temporary high concentrations with adverse effects.

Most amino acids elicit many basic tastes although one taste usually predominates. All D-amino acids, except proline and hydroxy proline, taste sweet and seven of the L-amino acids are sweet. D-proline is the only reportedly purely bitter D-amino acid and L-proline has a sweet-bitter taste, According to the invention both L and D-proline can be used, L-proline is preferred. Oral administration at relatively high doses and high concentrations of unpleasantly tasting compounds are to be avoided, in particular for the above mentioned reasons in individuals suffering from premature ageing syndromes that are generally difficult to nourish. Hence, slow release and/or encapsulated compositions according to the invention are preferred.

Premature or natural ageing symptoms to be delayed, prevented and/or treated according to this invention comprise, but are not limited to shortened life span, kyphosis, changes in body weight, low fat percentage (as determined by the fatty tissue vs. total body weight ratio) or fat redistribution, cachexia, hair loss, greying, neuronal and sensory dysfunction (loss of sight, hearing, smell, learning and memory capabilities), tremors, seizures, ataxia, sexual behaviour, fertility, muscle function, (limb-) coordination, heart function, hormonal-, immunological- or haematological-ageing parameters, telomere shortening, bone and skeletal disease such as osteoporosis or osteoschlerosis, retinal or macular degeneration, photoreceptor cell loss, liver dysfunction such as steatosis, kidney dysfunction, thymic involution, Purkinje-cell loss, anemia, immune dysfunction (including autoimmune disease), cardiovascular dysfunction, diabetes, and cancer in general as well as changes in gene expression patterns associated with ageing, as determined by RNA expression levels, protein expression levels, metabolite levels and hormone levels.

In a first aspect, the current invention provides the use of at least one of mannitol and proline for the manufacture of a medicament for delaying, preventing and/or treating (natural) ageing related symptoms. Preferably, in this medicament mannitol and/or proline are present in low concentrations as later exemplified herein. Medicaments according to this invention are particularly suitable for the treatment of human subjects suffering from premature ageing syndromes that may be caused by genetic defects in DNA repair pathways. Such premature ageing syndromes in humans comprise Cockayne syndrome (CS), Xeroderma pigmentosum (XP), combined XPCS, trichothiodystrophy (TTD), COFS (cerebro-oculo-facio-skeletal syndrome), XFE disorder (Xpf-Erccl syndrome), Bloom Syndrome (BS), Werner Syndrome (WS), Ataxia Telangiectasia (AT), Fanconi Anemia (FA), Hutchinson Guilford Progeria (HGP) and related genome maintenance syndromes.

In a second aspect, the invention provides compositions comprising as active constituents mannitol and/or proline. Mannitol is the preferred compound. Results obtained using a composition comprising both compounds may suggest a synergistic effect between the two. Although it may be convenient to administer mannitol and proline in one composition, both substances may also be administered in separate compositions simultaneously or sequentially to a human subject. Preferably, the composition is a pharmaceutical composition.

A combined composition according to the invention, comprising both mannitol and proline preferably has a molar ratio of mannitol to proline ranging from 1:10 to 10:1, most preferably between 1:3 to 3:1 respectively. The composition according to the invention may be liquid and is preferably solid or semi-solid. Usable concentrations of proline and mannitol depend on the body weight of the subject to be treated, the ageing related condition to be treated, the size of type of the pharmaceutical delivery system. The composition should preferably be such that the proline and/or mannitol levels in the tissues and bodily fluids of a subject to be treated are increased at least 50%, more preferably 100%, 200%, 300%, 500% or more, relative to an untreated individual. Preferably, the body fluid is serum. The free proline concentrations in the body range approximately from 1 to 100 microgram per ml. bodily fluid, depending on diet, condition and time of sampling. The mannitol concentrations in the body may vary, depending on the diet (fruits and vegetables are rich sources of mannitol) but generally range from undetectable to a maximum of approximately 5 microgram per ml. bodily fluid or gram tissue. Undetectable level of mannitol are preferably less than 0.025 μg/ml body fluid.

Accordingly in a preferred embodiment, a treated individual has a mannitol concentration in its scrum or effective mannitol concentration which is ranged between 0.0375 μg/ml and 75 μg/ml serum, more preferably between 0.04 and 10, 15, 20, 30 or more μg/ml serum. Accordingly, in a more preferred embodiment, the serum concentration or effective concentration in a treated individual is less than 10 mM mannitol, 10 mM mannitol is equivalent with 1800 μg/ml mannitol.

Accordingly in another preferred embodiment, a treated individual has a proline concentration in its serum which is ranged between 1.5 and 150 μg/ml serum.

The proline and mannitol concentrations are preferably assessed by LC-MS/MS (Liquid Chromatography/Mass Spectrometry). The presence of mannitol is preferably assessed in serum or whole blood samples after derivatisation and using ¹³C6 mannitol as internal standard. The LLOQ (Lowest Limit of Quantitation) for mannitol is typically 0.0250 μg/ml. The presence of proline is preferably assessed in serum samples with a concentration range of commercially available proline as internal standard. The LLOQ (Lowest Limit of Quantitation) for proline is typically 0.250 μg/ml. Preparation of the samples is preferably carried out as described in the examples.

Accordingly the current invention provides for new medicaments for use in the method of treatment according to this invention. Formulation of medicaments, ways of administration and the use of pharmaceutically acceptable excipients are known and customary in the art and for instance described in Remington; The Science and Practice of Pharmacy, 21^{nd} Edition 2005, University of Sciences in Philadelphia. The pharmaceutically (acceptable) composition according to the invention, comprising mannitol and/or proline may be formulated to be suitable for mucosal application. Alternatively, the composition may be formulated to be suitably used as an inhaler (spray) or as eyedrops or as suppositories, Pharmaceutical compositions and medicaments of the invention may comprise binders such as lactose, cellulose and derivatives thereof, polyvinylpyrrolidone (PVP), humectants, disintegration promoters, lubricants, disintegrants, starch and derivatives thereof, sugar solubilizers, immuno-stimulatory adjuvants or other excipients. The invention provides methods and means to formulate and manufacture new medicaments and/or pharmaceutical formulations for the treatment of (premature) ageing symptoms.

The compositions, preferably pharmaceutical compositions according to the invention are preferably controlled release formulations. More preferably, the controlled release formulation is a slow release formulation. The slow release formulation may be provided by a pump, slow dissolving coatings, polymers and/or fillers. The pump may be an osmotic pump. Such compositions can be formulated and adapted for routes of administration by any person skilled in the art of pharmacy and/or pharmacology. Compositions for slow release may be administered orally or subcutaneously to a subject to be treated.

For instance U.S. Pat. No. 4,880,830 shows slow release formulations to be administered to humans or animals, comprising primary granules which contain an active ingredient and are in a secondary matrix of a water soluble/dispersible slow release material, the granules themselves comprising particles containing the active ingredients and in a primary matrix of a water soluble/dispersible slow release material. Optionally, formulations comprises a binder phase of a water insoluble slow release material having embedded therein secondary granules comprising the secondary matrix containing the primary matrix granules. The water soluble/dispersible material may be a polysaccharide and acacia and low viscosity methylcellulose are exemplified, as well as alginate and gelatine.

The carrier vehicle for each component is selected from a wide variety of materials which are already known per se or may hereafter be developed which provide for controlled release of the compositions in the particular physiological environment. In particular, the carrier vehicle of the delivery system is selected such that near zero-order release of the components of the regimen is achieved. A targeted steady-state release can be obtained by suitable adjustment of the design or composition of the delivery system.

One suitable formulation to achieve the desired near zero-order release of the components comprises injectable microcapsules or microspheres prepared from a biodegradable polymer, such as poly(dl-lactide), poly(dl-lactide-co-glycolide), polycaprolactone, polyglycolide, polylactic acid-coglycolide, poly(hydroxybutyric acid), a polyortho-ester or a polyacetal.

Microcapsules are systems comprising a polymeric wall that encloses a liquid or solid core. The capsule wall usually does not react with the core material; however, it is designed to provide sufficient strength to enable normal handling without rupture while being sufficiently thin to allow a high core to wall volume ratio. The capsule contents remain within the wall until released by diffusion or other means that dissolve, melt, break, rupture or remove the capsule material. Preferably, the capsule wall can be made to degrade and decompose in suitable environments while diffusing the core material through the capsule wall to allow for its slow, prolonged delivery.

The mechanism of release in biodegradable microcapsules is a combination of drug diffusion and polymer biodegradation. Therefore, the rate and duration of release are determined by microcapsule size, drug content and quality, and polymer parameters, such as crystallinity, molecular weight and composition. In particular, adjustment in the amount of drug released is generally achieved by modification of capsule wall thickness, capsule diameter, or both. Detailed information concerning the design and use of microspheres and microcapsules is provided by, e.g., Lewis, D. H., “Controlled Release of Bioactive Agents from Lactide/Glycolide Polymers,” in Jason & Langer (eds.), Biodegradable polymers as drug delivery systems, pp. 1-41 (1990).

Alternatively or in combination with other exemplified controlled release formulation, a composition of the invention may be used in a pump. The pump can deliver appropriate quantities of a concentrated mannitol and/or proline containing composition in a sustainable fashion over days or weeks in order to reach serum concentrations of mannitol and/or proline as earlier indicated herein. Such pumps may be osmotic and/or infusion pumps. The skilled artisan is capable of choosing a pump from the wide range of pumps that are commercially available, with different infusion speeds, administration modes, capacities, accuracy etc. Accordingly, the invention in a further aspect provides a pump comprising a composition of the invention as herein defined.

In a composition according to the invention, mannitol is preferably present in a quantity of at least 0.5 percent by weight of the composition and proline is present in at least 0.5 percent by weight of the composition. More preferably, mannitol is present in a quantity which is ranged between 0.5 and 5% by weight of the composition and/or proline is present in a quantity which is ranged between 0.5 and 5% by weight of the composition. Even more preferably, mannitol is present in a quantity which is ranged between 1 and 4% by weight of the composition and/or proline is present in a quantity which is ranged between 1 and 4% by weight of the composition. Even more preferably, mannitol is present in a quantity which is ranged between 1.5 and 3% by weight of the composition and/or proline is present in a quantity which is ranged between 1.5 and 3% by weight of the composition. Most preferably, mannitol is present in a quantity of about 2% by weight of the composition and/or proline is present in a quantity of about 2% by weight of the composition.

These amounts are considered low and therefore attractive since low amounts of mannitol and/or proline were found to be especially effective and give less side effects (no exacerbation of the state of malnourishment for mannitol and/or no unpleasant taste for proline) as indicated earlier herein.

The pharmaceutically (acceptable) composition according to the invention, comprising mannitol and/or proline may also be formulated in an edible solid or liquid form such as a food composition, or a nutraceutical composition or formulation that is enriched for mannitol and proline. A nutraceutical or food composition according to this invention may be solid or liquid. Preferred liquid food compositions include a beverage and/or a dairy product. More preferred beverages include a soft drink. Preferred solid food compositions include a sweet, chips, an energy-bar or an enriched meal or meal-replacer or other fortified forms of food of beverage. The solid or liquid food compositions may comprise or may be derived from or may be based on each type of solid or liquid food compositions as exemplified above. The dairy product may be or may comprise or may be derived from milk, yoghurt or cheese. The softdrink may be carbonated. Alternatively, the softdrink is not carbonated. The sweet may be a chewing gum, or a liquorice. Alternatively, the composition is a cosmetic composition. Examples of cosmetic compositions include (sun) cream, lotion, shampoo, spray, dermal stick.

In a further aspect, the invention provides a method of treatment of a subject suffering from a premature ageing or segmental progeroid syndrome, comprising administering to a subject a source of mannitol and/or proline, preferably a slow release pharmaceutical composition according to the invention, in an amount effective to prevent, alleviate or cure one or more premature ageing symptoms in the subject treated.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The invention is further illustrated by the following examples, which should not be construed for limiting the scope of the invention.

### FIGURE LEGENDS

FIG. 1. Effect of mannitol on percentage Csb^{G744ter/G744ter}/Xpa^{null/null} pups born.

FIG. 2. Effect of mannitol on survival in weeks afterbirth for Csb^{G744ter/G744ter}/Xpa^{null/null} pups.

FIG. 3. Changes in the retina of Csb^{-/-} mice with age.

A) Micrographs were taken in the central part of the retina of 3-month-old Csb−/− (panel a), 18-month-old Csb−/− (panel b) and 18-month-old Csb+/+ mice (panel c). Note the loss of ONL nuclei and distortion of the outer segment layer in 18-month-old Csb−/− mice. Bar 25 μm. B) Counts of ONL nuclei (±standard deviation) demonstrate a loss of photoreceptor cells in Csb−/− mice with age. (ANOVA, followed by T-test; P<0.05), Xpa−/− mice at 6.5 months do not differ from wild type in photoreceptor number. C) Paraffin sections of 3 month-old wild type and Csb-deficient mice stained with a nuclear stain (DAP1) on the left and stained with FITC for TUNEL-positive cells on the right. Arrows point at TUNEL-positive nuclei in the ONL of the Csb−/− mouse retina.

### EXAMPLES

### Preparation of the Samples for Assessing the Presence of Mannitol or Proline by Lc-MS/MS

### Mannitol

The presence of mannitol is preferably assessed in serum samples after derivatisation using ¹³C6 mannitol as internal standard. The LLOQ (Lowest Limit of Quantitation) for mannitol is typically 0.0250 μg/ml. The sample is typically mixed with water and subsequently mixed with the standard spike solution. The sample is further precipitated using acetonitrile and centrifuged. After evaporation to dryness, the samples are derivatised and subsequently liquid-liquid extraction is performed. The organic layer is isolated and evaporated to dryness. The residue is dissolved in injection solvent and injected.

### Proline

The presence of proline is preferably assessed in serum samples using ¹³C15N-proline as internal standard. The LLOQ (Lowest Limit of Quantitation) for proline is typically 0.250 μg/ml. The sample is typically mixed with water and subsequently mixed with the standard spike solution. The sample is further precipitated using acetonitrile and centrifuged. An aliquot of the supernatant is evaporated to dryness under a steam of nitrogen at 60° C., Milli-Q water is added and the preparation is vortexed. Methanol is subsequently added to the preparation and vortexed. Usually Milli-Q water and methanol are added in a ratio of 1/5. The preparation is centrifuged for 2 minutes at a speed of 4000 g or more. The obtained preparation is injected. The apparatus used for LC-MS/MS is API4000 LC-MS/MS.

### Example 1

### Testing of Proline and Mannitol in NER Deficient Mice: Phenotypic Effects of Anti-Oxidants on Csb^{G744ter/G744ter}/Xpa^{null/null} Double Mutant Mice

This example shows in an experimental set-up that mannitol and proline can inhibit, prevent and/or delay genome maintenance induced symptoms, in particular ageing-related symptoms, in mice exhibiting mutations in NER/TCR pathways, thereby illustrating the usefulness of the method of screening compounds according to the current invention.

The mouse model used in this example was the CSB^{-/-}/XPA^{-/-} (double knock out, wherein Csb^{G744ter/G744ter}/Xpa^{null/null}) mouse model, exhibiting a defect in GG-NER and TC-NER (XPA^{-/-}) and TCR in general (CSB^{-/-}). CSB^{-/-} mice exhibit a mild ageing phenotype, a premature photoreceptor loss in the retina (example 3), while XPA mice are completely NER-defective but apart from strong cancer-predisposition and a slightly shorter life span fail to exhibit an overt phenotype to distinguish them from wild type mice. Interbreeding both mouse models however demonstrates that CSB^{-/-}/XPA^{-/-} (double mutant) mice are born in sub-mendelian frequencies, exhibit stunted growth, kyphosis, ataxia, cachexia, osteoporosis and generally die in the third week after birth. Additionally these animals have an enhanced photoreceptor cell loss. The accumulation of oxidative DNA damage before and immediately after birth presumably negatively influences transcription and causes the premature ageing phenotype.

In order to investigate the effect of radical scavengers on the CSB/XPA double knockout mice, the effect of several compounds and compositions was monitored by the frequency of CSB/XPA DKO mice (closer to the expected mendelian frequency of 25%), an extended life-span (longer than the average three weeks for untreated DKO mice) and a delay or to some extent inhibit the premature ageing phenotype.

To obtain CSB/XPA double mutant mice the following crossing were done:

(M) CSB^{-/-}XPA^{+/-}x(F) CSB^{-/-}XPA^{+/-}

(M) CSB^{-/-}XPA^{+/-}x(F) CSB^{+/-}XPA^{-/-}

(M) CSB^{+/-}XPA^{-/-}x(F) CSB^{+/-}XPA^{-/-}

(M) CSB^{+/-}XPA^{-/-}x(F) CSB^{-/-}XPA^{-/-}

From these crossings CSB^{-/-}XPA^{-/-} mice were born with a frequency of 9%, whereas the expected Mendelian frequency is 25%.

16 pregnant females received an osmotic pump, 7×30 mm, subcutaneously implanted under the skin on the back, for continuous release of Phosphate Buffered Saline (control) or 5% D-mannitol dissolved in Phosphate Buffered Saline. The offspring were genotyped following normal procedures (tail clipping and genomic DNA analysis by Southern blot analysis or PCR amplification) and monitored for life span.

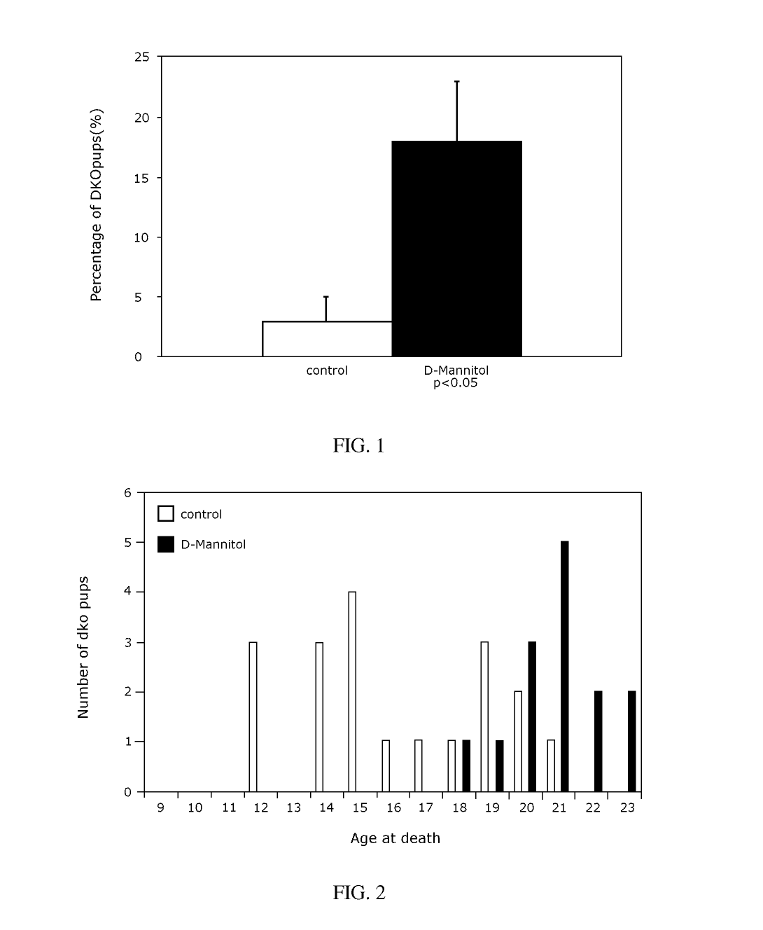

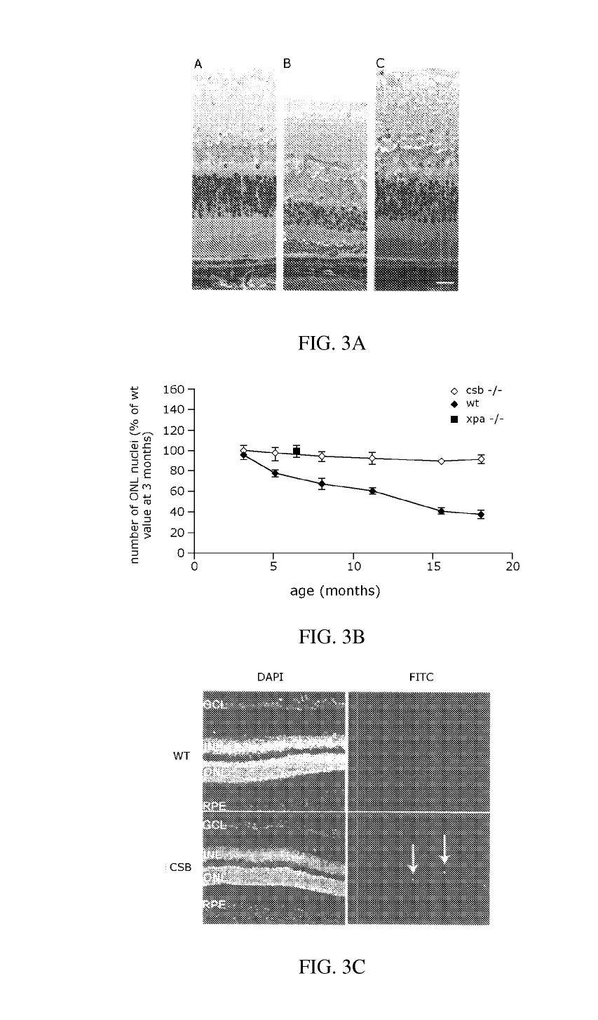

FIGS. 1 and 2 show the increase in frequency of birth of XPA/CSB double KO mice after treatment with hydroxyl scavenger D-mannitol (experiment 1) and the increase in survival after birth (life span) respectively. Comparable results were obtained in experiments in which 2% D-Mannitol was administered to drinking water. Comparable results were also obtained with another scavenger: proline, which was equally effective in increasing the frequency of survival of DKO pups after birth.

### Example 2

In order to measure the transmission of D-Mannitol and L-Proline from mother to pup via the placenta and/or mother milk, 20 pregnant wild type mice (C57B1/6) received an osmotic pump on day 3 after the detection of the postcoital plug, The osmotic pump (7×30 mm), subcutaneously implanted under the skin on the mouse's back, gave a continuous release of D-Mannitol and/or L-Proline, or Phosphate Buffered Saline (PBS) for the duration of 28 days. The osmotic pumps contained 200 μl of either 0.3M D-Mannitol, 0.3M L-Proline, or both 0.2M D-Mannitol and 0.2 M L-Proline, dissolved in Phosphate Buffered Saline. Each formulation was given to 5 females, and as a control 5 females received PBS. To determine the actual concentration of L-Proline and/or D-Mannitol present in the blood of the mothers, blood samples were taken every 7 days after the detection of the postcoital plug until day 42 (6 time points). For determination of transmission of these compounds through the placenta, embryo's were isolated from 1 mouse out of each treatment group at day 18.5, L-Proline and D-Mannitol contents were measured in whole tissue samples extracted from these animals. For determination of transmission of these compounds through the mother milk, mother milk from the treated mice and blood samples from the pups were collected at day 7, 14 and 21 after birth. Elevated levels of D-Mannitol or L-Proline in blood of mothers and pups, in milk or in whole embryo's were not detected, which is probably the result of tight endogenous regulation of L-Proline and D-Mannitol blood levels.

L-Proline was also measured in the blood and urine of four wt animals that received 0.2M L-Proline via their drinking water. As a control, four wt animals received just drinking water. The duration of this treatment was one week, after which one blood sample and one urine sample was taken. High levels of L-Proline were detected in the urine, but no elevated levels of L-Proline were detectable in the blood of these animals. This high renal clearance of L-proline again shows that L-Proline levels in blood are tightly regulated.

### Example 3

CSB mice have accelerated aging-related photoreceptor loss. This can be shown by TUNEL staining of the retina, which reveals apoptotic cells. The photoreceptor loss can also be induced in young adult CSB mice (8-10 weeks) with γ-irradiation (induces a.o. oxidative DNA damage). Since aging-related photoreceptor-loss is at least in part caused by unrepaired oxidative DNA damage, we are able to intervene with anti-oxidant or radical scavenger molecules, in particular with mannitol and/or proline.

21 CSB animals, age 6 months, received an osmotic pump, 7×30 mm (volume 200 μl), subcutaneously implanted under the skin on the back, for continuous release of Phosphate Buffered Saline (control), 0.3M D-Mannitol or 0.03M D-Mannitol dissolved in Phosphate Buffered Saline, 7 animals per compound. To determine the actual D-Mannitol concentration in the blood of these animals, blood samples were taken every 7 days after the implantation of the osmotic pump. After three weeks, eyes were isolated from these animals and TUNEL staining was performed to determine the effect of D-Mannitol on diminishing the age-related photoreceptor-loss in CSB animals, Blood and urine samples were taken for measurements of D-mannitol levels and for determination of biomarkers. The results are depicted in the third column of table 1. In a second experiment, the D-Mannitol treatment via osmotic pumps were repeated, but here on 8 week-old CSB mice were γ-irradiated, in order to see if D-mannitol can reduce the γ-induced photoreceptor-loss observed in CSB mice. The results are depicted in the fourth column of table 1.

For retinal evaluation by TUNEL staining, eyes were marked nasally with Alcian blue (5% Alcian blue in 96% ethanol), enucleated, fixed in 4% paraformaldehyde in 0.1M phosphate buffer, washed in PBS and embedded in paraffin. Horizontal sections (5 μm thick) of the retina were cut and sections in the middle of the retina were selected by Alcian blue marking and proximity of the optic nerve. Sections were stained for degenerating cells by TdT-mediated dUTP Nick-End Labeling (TUNEL), according to the manufacturer's instructions (Apoptag Plus Peroxidase In Situ Apoptosis Detection Kit, Chemicon). For quantification, the number of TUNEL-positive cells in the inner nuclear layer (INL) and outer nuclear layer (ONL) were counted in 6 whole sections per mouse.

Wild type and Csb^{m/m} mice (8-10 week old; n=6) received a brief (13 min) total body irradiation of 10 Gy using a ¹³⁷Cs source. After 20 hrs, animals were sacrificed and eyes were processed for further histopathological analysis.

TABLE 1
Protective effect of mannitol on photoreceptor loss

| | Serum | | |
|---|---|---|---|
| | [mannitol] | Apoptotic | Apoptotic |
| | after 3 weeks | index¹ | index¹ |
| Treatment | (μg/ml) | without radiation | after radiation |
| PBS (control) | 0.01 ± 0.03 | 33.1 ± 7.8 | 52.4 ± 9.1 |
| 0.03M Mannitol | 0.10 ± 0.03 | 24.1 ± 6.8 | 29.5 ± 6.0 |
| 0.3 Mannitol | 0.65 ± 0.18 | 24.8 ± 6.0 | 28.9 ± 11.3 |

¹Based on the number of TUNEL-positive cells, a higher number denotes higher levels of apoptosis.

As can be seen in table 1, treatment with mannitol resulted in a significant decrease of the apoptotic index, which is indicative of a decrease of photoreceptor loss. This effect was observed as a result of either endogenous oxidative stress (third column of table 1) or exogenous (radiation induced) oxidative stress (fourth column of table 1).

What is claimed is:

1. A method for delaying, preventing and/or treating ageing related symptoms in an individual, the method comprising administration of a medicament comprising mannitol to the individual, wherein an effective mannitol concentration in the treated individual is about 10 mM mannitol or less.

2. The method of claim 1, wherein the individual suffers from premature ageing syndrome and/or genetic defects in a DNA damage response/repair pathway.

3. The method of claim 2, wherein the individual suffers from premature ageing syndrome and the premature ageing syndrome is selected from the group consisting of Cockayne syndrome (CS), Xeroderma pigmentosum (XP), combined XPCS, trichothiodystrophy (TTD), COFS (cerebro-oculo-facio-skeletal syndrome), XFE disorder (Xpf-Erccl syndrome), Bloom Syndrome (BS), Werner Syndrome (WS), Ataxia telangiectasia (AT), Fanconi Anemia (FA), Hutchinson Guilford Progeria (HGP), and related genome maintenance disorders.

4. The method of claim 1, wherein the medicament further comprises proline and wherein the medicament is a controlled release formulation.

5. The method of claim 4, wherein the controlled release formulation is provided by a pump, slow dissolving coatings, polymers and/or fillers.

6. (canceled)

7. The method of claim 4, wherein the medicament comprises mannitol and proline in a molar ratio of 1:10 to 10:1.

8. A composition for use in treating an individual, wherein the composition comprises mannitol, and wherein the amount of mannitol is sufficient to produce a concentration in a treated individual of about 10 mM or less.

9. The composition according to claim 21, wherein mannitol and proline are in a molar ratio of 1:10 to 10:1.

10. The composition according to claim 8, wherein the composition is a controlled release formulation.

11. The composition according to claim 10, wherein the controlled release formulation is provided by slow dissolving coatings, polymers and/or fillers.

12. The composition according to claim 21, wherein mannitol is present in a quantity ranged between 0.5 and 5 percent by weight of the composition and proline is present in a quantity ranged between 0.5 and 5 percent by weight of the composition.

13. The composition according to claim 8, wherein the composition is a pharmaceutical composition.

14. The composition according to claim 8, wherein the composition is a nutraceutical composition.

15. The composition according to claim 14, wherein the composition comprises a beverage, a dairy product, a sweet, chips, an energy-bar, an enriched meal or meal-replacer, or other fortified forms of food or beverage.

16. The composition according to claim 8, wherein the composition is a cosmetic composition.

17. A pump comprising the composition as defined in claim 8.

18. A pump comprising the composition as defined in claim 13.

19. A method for delaying, preventing, and/or treating ageing related symptoms in an individual, the method comprising administration of a medicament comprising proline to the individual.

20. The method of claim 19, wherein the medicament further comprises mannitol, and wherein an effective mannitol concentration in the treated individual is about 10 mM mannitol or less.

21. The composition of claim 8, wherein the composition further comprises proline.
